SEQUENCE LISTING (3 sequences)

SEQ ID NO: 1 - protein, 217 aa, Saccharomyces cerevisiae
MSWRYALKNYVTSPETVNDDTVTYFDDKVSIIRDSFPKSECHLLILPRTMQLSRSHPTKVIDAKFKNEFESYVNSAIDHIFRHFQEKFRIKKSDDDKDPCWDDILKDKNKFVRNFVQVGIHSVPSMANLHIHVISKDFHSVRLKNKKHYNSFNTGFFISWDDLPLNGKNLGTDKEIETTYLKEHDLLCCYCQRNFSNKFSLLKKHLELEFNSHFELK

SEQ ID NO: 2 - protein, 64 aa, Artificial Sequence
  Synthetic construct
PCGATGGGACCTACAATGTACCAGAAGCGTCFAMGCTACCCTGGATGTTACATGGTCTTCGCAG

SEQ ID NO: 3 - protein, 342 aa, Homo sapiens
MMRVCWLVRQDSRHQRIRLPHLEAVVIGRGPETKITDKKCSRQQVQLKAECNKGYVKVKQVGVNPTSIDSVVIGKDQEVKLQPGQVLHMVNELYPYIVEFEEEAKNPGLETHRKRKRSGNSDSIERDAAQEAEAGTGLEPGSNSGQCSVPLKKGKDAPIKKESLGHWSQGLKISMQDPKMQVYKDEQVVVIKDKYPKARYHWLVLPWTSISSLKAVAREHLELLKHMHTVGEKVIVDFAGSSKLRFRLGYHAIPSMSHVHLHVISQDFDSPCLKNKKHWNSFNTEYFLESQAVIEMVQEAGRVTVRDGMPELLKLPLRCHECQQLLPSIPQLKEHLRKHWTQ

(19) United States
(12) Patent Application Publication
(10) Pub. No.: US 2015/0218608 A1
(43) Pub. Date: Aug. 6, 2015

(54) Compositions and Methods for Improvement of Ligation Yields

(71) Applicant: NEW ENGLAND BIOLABS, INC., Ipswich, MA (US)

(72) Inventors: Gregory Lohman, Cambridge, MA (US); Nicole Nichols, Reading, MA (US)

(21) Appl. No.: 14/608,864

(22) Filed: Jan. 29, 2015

Related U.S. Application Data
(60) Provisional application No. 61/934,251, filed on Jan. 31, 2014.

(51) Int. Cl. C12P 19/34; C12N 9/00; C12N 9/22

(52) U.S. Cl. CPC C12P 19/34; C12N 9/22; C12N 9/93; C12Y 605/01001; C12Y 301/13004

(57) ABSTRACT

Methods and compositions are provided for increasing the ligation yields of polynucleotides in vitro.

### CROSS REFERENCE

This application claims right of priority to provisional patent application Ser. No. 61/934,251, filed Jan. 31, 2014.

### BACKGROUND

In vitro methods for the joining of polynucleotides (DNA and RNA) depend on a ligase to catalyze the ligation of a terminal phosphate on one polynucleotide to a 3′-hydroxyl terminus of another polynucleotide, or the circularization of a single polynucleotide with both a 5′-phosphate (5′P) and a 3′-hydroxyl terminus (Tomkinson, et al., Chem. Rev., 106(2): 687-699 (2006); Ellenberger, et al., Annu. Rev. Biochem., 77:313-338 (2008); Pascal, Curr. Opin. Struct. Biol., 18(1):96-105 (2008); Shuman, J. Biol. Chem., 284(26): 17365-17369 (2009). ATP and NAD⁺ dependent ligases may ligate substrates with base pair mismatches, gaps of one or more nucleotides between the ligatable ends, oligonucleotides containing non-standard bases, and other complex structures with varying efficiencies (Olivera, et al., PNAS, US, 61(1):237-244 (1968); Harvey, et al., J. Biol. Chem., 246(14): 4523-4530 (1971); Nilsson, et al., Nucleic Acids Res., 10(5):1425-1437 (1982); Zimmerman, et al., Proc. Natl. Acad. Sci. USA, 80(19):5852-5856 (1983); Goffin, et al., Nucleic Acids Res. 15(21): 8755-8771 (1987); Wiaderkiewicz, et al., Nucleic Acids Res., 15(19): 7831-7848 (1987); Wu, et al., Gene, 76(2): 245-254 (1989); Pritchard et al., Nucleic Acids Res., 25(17):3403-3407 (1997); Cherepanov, et al., J. Biochem., 129(1):61-68 (2001); Chiuman, et al., Bioorg. Chem. 30(5): 332-349 (2002); Nandakumar, et al., Mol. Cell, 26(2):257-271 (2007); Patel, et al., Bioorg. Chem., 36(2):46-56 (2008)). Many of these polynucleotides have been shown to produce a mixture of ligated product and 5′-adenylylphosphorylated (5′App) polynucleotide; where the 5′App polynucleotides can be dead end products or react only extremely slowly in the presence of the ATP and NAD⁺ cofactors found in most ligase buffers.

### SUMMARY

In general in one aspect, compositions and methods are provided for improving the ligation yields of polynucleotides prone to producing 5′App DNA in the presence of a ligase.

In general in one aspect, an in vitro ligation mixture is provided that includes a 5′-deadenylase (excluding human aprataxin, such as SEQ ID NO:3) such as a yeast 5′-deadenylase or variants thereof such as S. cerevisiae HNT3 (see for example, Daley, et al., DNA Repair, 9(6):690-699 (2010) (see for example SEQ ID NO:1) or variants (for example, those having at least 90% amino acid sequence identity to SEQ ID NO:1), an isolated polynucleotide ligase such as a DNA or RNA ligase such as T4 DNA ligase and one or more polynucleotides, wherein one or more polynucleotides have a 5′App or 5′P, in a buffer containing at least 10 μM ATP and/or 10 μM NAD⁺ preferably at a pH greater than pH 7.0, for example, at least pH 7.3, pH 7.4, pH 7.5, pH 7.6 or pH 7.7.

Optionally the 5′-deadenylase is a non-naturally occurring enzyme. Optionally the 5′-deadenylase differs from the wild type 5′-deadenylase by at least one amino acid. Optionally, the ligase differs from the wild type ligase by at least one amino acid.

In general in one aspect, an in vitro method is provided for enhancing the yields of ligation of polynucleotide, the method including (a) in a reaction vessel, incubating an in vitro ligation mixture such as described above and a plurality of polynucleotides, wherein one or more of the polynucleotides have a 5′P and/or 5′App, and wherein the ligation mixture may additionally contain a ligation enhancer such as a small ligation enhancer or PEG or Ficoll and may have a pH greater than pH 7; and (b) enhancing yields of ligated polynucleotides.

The following description of component enzymes and polynucleotides in the mixture applies to both compositions and methods.

Whereas T4 DNA ligase is described above as an example of a ligase, the activity of other ligases may also be improved as described herein by adding a 5′-deadenylase. These include for example, T3 DNA ligase, T7 DNA ligase, T4 RNA ligase 1, T4 RNA ligase 2, T4 RNA ligase 2 (truncated), T4 RNA ligase 2 (truncated KQ), PBCV-1 DNA ligase, E. coli DNA ligase, Taq DNA ligase, Mth ligase, or 9° N DNA ligase.

Examples of a 5′-deadenylase for use herein include a protein having at least 90% sequence identity to SEQ ID NO:1. In one embodiment, human aprataxin is excluded (SEQ ID NO:3). In another embodiment, a protein that is similar to SEQ ID NO:3 having 99%, 98%, 97%, 96%, 95%, 94%, 93%, 92%, 91% or 90% sequence identity to SEQ ID NO:3 may be excluded from embodiments of the composition or methods described herein.

In another aspect, the reaction buffer comprises Tris or Tricine buffer. In one aspect, the buffer has a pH greater than the pH of the cytoplasm or nucleus of a cell for example, greater than pH 7.0, pH pH 7.2, pH 7.3, pH 7.4, or pH 7.5. In addition, the buffer may include glycerol and/or a reducing agent such as DTT or TCEP. T4 DNA ligase buffer is an example of a suitable buffer. In another aspect, the reaction buffer contains a ligation enhancer. In another aspect, the ligation enhancer is a small molecule enhancer such as described in for example, U.S. Pat. No. 8,697,408. In another aspect, the ligation enhancer is a large molecule enhancer selected from the group consisting of PEG and Ficoll.

In another aspect, the one or more polynucleotides may initially contain a 5′P but during the ligation reaction, the polynucleotide 5′ terminal is converted to 5′App that would normally be resistant to completion of the ligation reaction. Hence a ligation mixture which was initiated with polynucleotides all containing a 5′P may become a ligation mixture in which some of the polynucleotides have a 5′App. Hence the embodiments include an in vitro ligation mixture wherein one or more polynucleotides have a 5′App. The ligation mixture in this context refers to the ligation mixture that is present during a reaction in which all molecules may initially have a 5′P.

The one or more polynucleotides in a ligation mixture may include at least one of the following: a nicked polynucleotide molecule with two abutting oligonucleotides annealed to a complementary polynucleotide splint where the ligase is a splint ligase (see for example, US 2014/0179539); a polynucleotide annealed to a complementary polynucleotide splint such that there is a 1, 2, 3 or 4 nucleotide (nt) gap between ligatable ends; a nicked polynucleotide with two abutting oligonucleotides annealed to a complementary polynucleotide splint with one or more mismatched base pairs near the ligation junction; a nicked polynucleotide with two abutting oligonucleotides annealed to a complementary DNA splint with one or more non-standard bases near the ligation junction; polynucleotides with short complementary overhangs (cohesive ends) or with fully base paired (blunt end) termini; a polynucleotide with short complementary overhangs (cohesive ends) or with fully base paired (blunt end) termini incorporating mismatched base pairs or non-standard nts near the site of ligation, and/or a single polynucleotide which when ligated forms a circle. In another aspect, an example of a non-standard base is inosine.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows the results of ligating a blunt-end double stranded DNA (dsDNA) using a ligase in the presence of a 5′-deadenylase compared with a ligase in the absence of a 5′-deadenylase under the same conditions with the same components except for 5′-deadenylase. The open circles show the yield of ligation product over time with no added 5′-deadenylase; yield reaches a maximum of about 33% around 45 minutes of incubation, with the balance of the material converted to unreactive 5′App DNA. The x shows the ligation yield when 40 units of recombinant yeast 5′-deadenylase (New England Biolabs, Ipswich, Mass.) is included with the T4 DNA ligase. The ligation yield continues to increase with prolonged incubation with a yield greater than 75% at 3 hours. No App DNA could be detected in this reaction.

### DETAILED DESCRIPTION OF THE EMBODIMENTS

Before various embodiments are described in greater detail, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

The in vitro ligation yield for any substrate prone to producing 5′App DNA as a side product can be substantially enhanced by use of a 5′-deadenylase in the in vitro ligation mixture. Enhanced ligation may refer to higher yields of ligation product than can be achieved with the same amount of ligase absent the 5′-deadenylase. While not wishing to be limited by theory, the 5′-deadenylase may convert 5′App DNA side products to a ligation-competent 5′P through enzymatic cleavage of the 5′App group from the 5′ end of DNA or RNA. In combination with a ligase, a complete reaction of ligation polynucleotides can occur under conditions that normally produce large amounts of unreactive 5′App DNA as a dead end product. The ability to enhance in vitro ligation and thus use less substrate and enzyme to obtain the same yield of product has utility for all uses of a ligation reaction in molecular biology. Such uses include one or more of the following examples: adding adaptors to target polynucleotides prior to amplification, adding modified nts, circularizing polynucleotides, joining single strand oligonucleotides on a splint oligonucleotide, polynucleotide repair and sequencing reactions.

The term “non-naturally occurring” refers to a composition that does not exist in nature.

In the context of a nucleic acid, the term “non-naturally occurring” refers to a nucleic acid that contains: a) a sequence of nts that is different to a nucleic acid in its natural state, b) one or more non-naturally occurring nt monomers (which may result in a non-natural backbone or sugar that is not G, A, T or C) and/or C) may contain one or more other modifications (e.g., an added label or other moiety) to the 5′-end, the 3′-end, and/or between the 5′- and 3′-ends of the nucleic acid.

In the context of a protein, the term “non-naturally occurring” refers to a protein that has an amino acid sequence and/or a post-translational modification pattern that is different to the protein in its natural state. For example, a non-naturally occurring protein may have one or more amino acid substitutions, deletions or insertions at the N-terminus, the C-terminus and/or between the N- and C-termini of the protein. A “non-naturally occurring” protein may have an amino acid sequence that is different to a naturally occurring amino acid sequence but that that is at least 80%, at least 85%, at least 90%, at least 95%, at least 97%, at least 98% or at least 99% identical to a naturally occurring amino acid sequence. In certain cases, a non-naturally occurring protein may contain an N-terminal methionine or may lack one or more post-translational modifications (e.g., glycosylation, phosphorylation, etc.) if it is produced by a different (e.g., bacterial) cell.

In the context of a composition or preparation, the term “non-naturally occurring” refers to: a) a combination of components that are not combined by nature, e.g., because they are at different locations, in different cells or different cell compartments; b) a combination of components that have relative concentrations that are not found in nature; c) a combination that lacks something that is usually associated with one of the components in nature; d) a combination that is in a form that not found in nature, e.g., dried, freeze dried, crystalline, aqueous; e) a combination that contains a component that is not found in nature. For example, a preparation may contain a buffering agent (e.g., Tris, HEPES, TAPS, MOPS, tricine or MES), a detergent, a dye, a reaction enhancer or inhibitor, an oxidizing agent, a reducing agent, a solvent or a preservative that is not found in nature and/or (f) the combination is contained in a non-cell container such as a reaction vessel, where the term reaction vessel refers to a tube, or well in which reagents may be in solution or immobilized where immobilization may occur on the surface of the reaction vessel or on a bead in the reaction vessel.

The enzymes used in the reaction may be purified by means of an affinity tag which may remain on the reagent after purification without interfering with the reaction. Examples of affinity tags include maltose binding protein (MBP), chitin binding domain (CBD), HisTag, SNAP-Tag® (New England Biolabs, Ipswich, Mass.), GST or any other tag known in the art. The reaction may be performed in solution. The reaction may be performed with one or more immobilized reagents where immobilization may include beads for example.

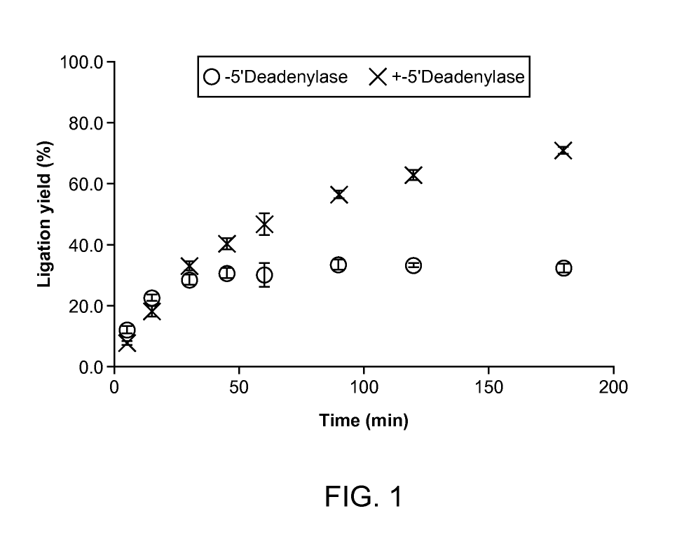

While not wishing to be limited by theory, the maximum achievable ligation yield may be achieved for any substrate that produces a mixture of ligation product and 5′App DNA upon incubation with a ligase by recycling 5′App polynucleotide side products formed. Continued incubation allows the ligase to react with a large majority of the substrate to the ligated structure. Inclusion of 5′-deadenylase with the ligase will allow enhanced yields of ligation product over a defined time period compared with ligation products obtained in the absence of the 5′-deadenylase. Enhanced yields may also be observed in the presence of 5′-deadenylase using incubation times longer than that at which the ligation reaction in the absence of 5′-deadenylase reaches a maximum yield. FIG. 1 shows yields at about 30 minutes incubation are similar to about 30% yield of ligation product under the conditions described in Example 1. Thereafter in the absence of 5′-deadenylase, ligation yields are constant while in the presence of 5′-deadenylase, yields of about 80% ligation are achieved in less than 200 minutes after the start of the reaction. The ligation product yield will continue to increase with prolonged incubation with 5′-deadenylase and ligase.

In aspects of the method, 1-5 units of 5′-deadenylase may be added to a 50 μL reaction with 10 nM-1 μM ligase concentration to cause enhanced ligation with for example, increased yields in an amount for example of >25%, >50%, >55%, >60%>65%>70%>75%>80%>85%>90%>100%>150%>200%>300% or as much as 400% enhancement over an incubation time period greater than 5 minutes, 10 minutes, 20 minutes, 40 minutes, 60 minutes, 80 minutes, 100 minutes, or 150 minutes or as much as 24 hours, 22 hours, 20 hours, 18 hours or 16 hours. Such comparison may be performed under similar or identical conditions except with respect to the present or absence of 5′-deadenylase thus demonstrating the efficacy of the 5′-deadenylase described herein (excluding human aprataxin) for enhancing the yield of ligation. As much as 99%, 97%, 95%, 93%, 91%, 89%, 87%, 85%, 80%, 75% or 70% ligation of substrate can be observed using the reaction mixture containing ligase and 5′-deadenylase after a suitable incubation time as described herein.

The 5′-deadenylase includes any non-human 5′-deadenylase for example a 5′-deadenylase having at least 90% sequence identity to yeast 5′-deadenylase (SEQ ID NO:1) or alternatively, or in addition, a 5′-deadenylase which has less than any % amino acid sequence identity in the range of 90%-99% to human aprataxin (SEQ ID NO:3).

In one embodiment, a unit definition for the 5′-deadenylase is the amount of enzyme required to remove 10 pmoles of AMP from a 5′-adenylated DNA oligo in 10 minutes at 30° C. In this example, an amount of 5′-deadenylase for enhancing ligation may include 1 unit 5′-deadenylase to 250 units T4 DNA Ligase (or 50 units to 1000 units).

Ligases for use in embodiments of the method include any polynucleotide ligase, T3 DNA ligase, T7 DNA ligase, T4 DNA ligase, T4 RNA ligase 1, T4 RNA ligase 2, T4 RNA ligase 2 (truncated), T4 RNA ligase 2 (truncated KQ), PBCV-1 DNA ligase, E. coli DNA ligase, Tag DNA ligase, Mth ligase, or 9° N DNA ligase.

In one embodiment, a unit definition for a DNA ligase is the amount of DNA ligase enzyme required to give 50% ligation of HindIII fragments of λ DNA (5′ DNA termini concentration of 0.12 μM, 300 μg/ml) in a total reaction volume of 20 μl in 30 minutes at 16° C. in 1×T4 DNA Ligase Reaction Buffer (New England Biolabs, Ipswich, Mass.).

In one embodiment, a unit definition for an RNA ligase is the amount of RNA ligase enzyme required to convert 1 nmol of 5′-³²P rA₁₆ into a phosphatase-resistant form in 30 minutes at 37° C.

Non-standard DNA bases can be joined by the ligase to the polynucleotide in the presence of a 5′-deadenylase such as in nick ligation using a splint oligonucleotide. Examples of non-standard DNA bases include inosine, α-thiotriphosphate containing the nucleobase P, pseudothymidine, difluorotoluene, substitutions in the furanose ring such as α-(L)-threofuranosyl-(3′-2′) nucleic acid (TNA), extra methylene group covalently linked to C4′ and the oxygen of C2′ of the furanose ring (LNA), nucleic acid analogs such as hexitol nucleic acid (HNA), flexible nucleic acid (FNA) such as formyl glycerol enantiomer, peptide nucleic acid such as aminoethylglycine PNA and thioester PNA.

The ligation reaction buffer may be any standard ligase reaction buffer, for example buffers with pH 7-pH 9, Mg⁺⁺ concentration 0.5 mM-20 mM, 0 mM-2 mM ATP or 0 mM-2 mM NAD⁺, buffers optionally containing large macromolecular enhancers (for example, PEG-6000, PEG-8000, Ficoll 70) in concentrations of 0-15 wt %, and/or buffers containing non-enzyme small molecule enhancers (see for example, U.S. Pat. No. 8,697,408) from 0-20 wt %. In one embodiment, the reaction conditions are 100 nM of a blunt-end dsDNA substrate using 10,000 U T4 DNA ligase in a 100 μL reaction volume with 50 mM Tris pH 7.5, 10 mM MgCl₂, 10 mM DTT, 1 mM ATP at 25° C. (see for example, FIG. 1).

A “polynucleotide” refers to double strand (ds) or single strand (ss) DNA or RNA or partially DNA and partially RNA for example where one strand is DNA and a complementary strand is RNA or where a polynucleotide strand may be partially DNA and partially RNA. The polynucleotide may also be nicked for example a double stranded nucleic acid containing mismatched base pairs, gaps of 1 or more nts at the ligation junction, or non-standard nts. A plurality of polynucleotides may include one or more of the following: two double stranded oligonucleotides with cohesive ends, single base overhangs or blunt ends; two single stranded oligonucleotides; one double stranded structure and one single stranded oligonucleotide. A single polynucleotide for ligation includes a single double strand or single strand oligonucleotide that is circularized upon ligation. The polynucleotides may be DNA, RNA, or any combination. Difficult to ligate polynucleotides are substrates that require any combination of high ligase concentration or buffers containing enhancers such as PEG or small molecule enhancers (see above) to achieve moderate to high ligation yields. Difficult to ligate polynucleotides are also polynucleotides that produce a mixture of 5′App DNA and ligation product upon initial incubation with a DNA ligase.

A ligase/5′-deadenylase/polynucleotide mixture under the conditions described herein can be used to achieve high ligation yields for any difficult to ligate substrate that produces a mixture of App DNA and ligated product when incubated with ligase alone. Examples of polynucleotide ligation reactions include intermolecular ligation of dsDNA or dsRNA substrates with blunt ends or short cohesive ends, with or without base-pair mismatches 0 nt-4 nts from the ligation junction; ligation between ssDNA and ssRNA polynucleotides; ligation between dsDNA or dsRNA polynucleotides or both containing a single nicked strand with mismatched base pairs or non-standard nts near the ligation junction; ligation of dsDNA or dsRNA polynucleotides containing a gap of 1 or more nt (for example 1, 2, 3, or 4 nt) between the 3′-hydroxyl and 5′-phosphorylated strands, or single, double stranded or partially double stranded or single stranded polynucleotides for circularization by ligation.

Some examples of uses of the present methods and compositions include DNA repair, DNA cloning, DNA sequencing, library preparation, RNA synthesis, miRNA and siRNA etc. and other methods known in the art such as described in the 2013/14 New England Biolabs Catalog.

### EXAMPLES

### Example 1

### Enhanced Ligation Yields for the Ligation of Blunt Ends and Single Base Overhangs

In a 100 μL final reaction volume, 5 μL of high concentration T4 DNA ligase (New England Biolabs, Ipswich, Mass.), 10 μL of a 1 μM stock of FAM-labeled, blunt-ended dsDNA substrate (e.g. SEQ ID NO:2).

SEQ ID NO: 2:
5′-pCGA TGG GAC CTA CAA TGT ACC AGA AGC
GTC-FAM-3′
3′-GCT ACC CTG GAT GTT ACA TGG TCT TCG CAG

10 μL of 10×T4 DNA ligase reaction buffer and either 0 μL or 2 μL of 5′-deadenylase (New England Biolabs, Ipswich, Mass.) were combined and incubated at 25° C. for 3 hours. 5 μL aliquots were removed throughout the incubation time and quenched with 12 μL stop solution (50 mM EDTA, 0.1% Triton X-100) then diluted with 500 μL ddH2O. The GeneScan™ 120 LIZ® Size Standard was diluted 1:40 in formamide (Applied Biosystems, Carlsbad, Calif.) and 10 μl of this solution combined with 1 μL of each sample before application to either a 3730xl Genetic Analyzer (96 capillary array) (Applied Biosystems, Carlsbad, Calif.) at a 36 cm capillary length with POP7 polymer. Data was collected via Applied Biosystems Data Collection software and analyzed using PeakScanner™ software (V 1.0) (Applied Biosystems, Carlsbad, Calif.). The retention times and areas of all peaks in the blue (FAM) channel were recorded. Oligonucleotides (phosphorylated starting material, adenylated intermediate, and ligation product) were identified by co-elution with synthetic standards. The fraction of each oligonucleotide in the sample was determined by dividing the peak area of each by the total peak area of all three oligonucleotides. The results are shown in FIG. 1.

### Example 2

### Enhanced Ligation Yields for the Ligation of Blunt Ends and Single Base Overhangs Using a Non-Naturally Occurring 5′-Deadenylase

A library of variant 5′-deadenylases that differed from the wild type by at least one amino acid are created using standard methods known in the art for random mutagenesis such as error prone PCR (see for example, Pritchard, et al., Journal of Theoretical Biology, 234:497-509 (2005)). The variants are screened in the assay described in Example 1 and the best performing 5′-deadenylases are selected for use to enhance ligation of two polynucleotides as described above.

### Example 3

### Enhanced Ligation Yields for the Ligation of Blunt Ends and Single Base Overhangs Using a Non-Naturally Occurring Ligase

Using a similar standard method of random mutagenesis, a ligase may be modified by changing at least one amino acid normally present and screening for activity as described in Example 2.

All references cited herein including provisional patent application Ser. No. 61/934,251, filed Jan. 31, 2014, are incorporated by reference.

What is claimed is:

1. An in vitro ligation mixture comprising:
at least 10 μM ATP and/or at least 10 μM NAD⁺;
a 5′-deadenylase protein that is not human aprataxin;
a polynucleotide ligase;
one or more polynucleotides, wherein the one or more of the polynucleotides has a 5′-phosphate (5′P) or adenylylphosphorylated (5′App); and
a buffering agent.

2. The mixture according to claim 1, wherein the 5′-deadenylase is a yeast 5′-deadenylase or a variant thereof and the ligase is from a source other than yeast.

3. The mixture according to claim 1, wherein the ligase is a DNA ligase.

4. The mixture according to claim 1, wherein the mixture has a pH greater than pH 7.0.

5. The mixture according to claim 1, where the ligation mixture comprises a ligation enhancer.

6. The mixture according to claim 5, wherein the ligation enhancer is selected from the group consisting of a small molecule enhancer, PEG and Ficoll.

7. The mixture according to claim 1, wherein the one or more polynucleotides comprise a nicked polynucleotide with two abutting oligonucleotides annealed to a complementary polynucleotide splint.

8. The mixture according to claim 1, wherein the one or more polynucleotides comprise short complementary overhangs (cohesive ends) or fully base paired (blunt end) termini.

9. The mixture according to claim 1, wherein the one or more polynucleotides comprise two polynucleotide molecules with short complementary overhangs (cohesive ends) or with fully base paired (blunt end) termini incorporating mismatched base pairs or non-standard nucleotides at or near the site of ligation.

10. A method for enhancing the yield of ligation of polynucleotides, the method comprising:
(a) in a reaction vessel, incubating an in vitro ligation mixture according to claim 1; and
(b) enhancing the yield of ligated polynucleotides.

11. The method according to claim 10, wherein the ligase is T4 DNA ligase.

12. The method according to claim 10, wherein the 5′-deadenylase has at least 90% sequence identity to SEQ ID NO:1.

13. The method according to claim 10, wherein the mixture has a pH is greater than pH 7.0.

14. The method according to claim 10, where the ligation mixture contains a ligation enhancer.

15. The method according to claim 14, wherein the ligation enhancer is selected from the group consisting of a small molecule enhancer, PEG and Ficoll.

16. The method according to claim 10, the method results in an increase in yield of ligation of ligation product by greater than 25% compared to the yield in the absence of the 5′-deadenylase for corresponding samples after 45 minutes of incubation.

17. The method according to claim 10, the method results in an increase in yield of ligation of ligation product by greater than 50% compared to the yield in the absence of the 5′-deadenylase for corresponding samples after 45 minutes of incubation.
